(19) United States
(12) Patent Application Publication
(10) Pub. No.: US 2021/0244550 A1
(43) Pub. Date: Aug. 12, 2021

(54) Silicone prosthesis provided with elements for attachment and use thereof

(71) Applicant: Pedro Alexandre DA MOTTA MARTINS, Porto Alegre (BR)

(72) Inventor: Pedro Alexandre DA MOTTA MARTINS, Porto Alegre (BR)

(21) Appl. No.: 17/245,878

(22) Filed: Apr. 30, 2021

Related U.S. Application Data
(63) Continuation of application No. PCT/BR2019/050427, filed on Oct. 1, 2019.

(30) Foreign Application Priority Data
Oct. 30, 2018 (BR) BR102018072311A2

(51) Int. Cl. A61F 2/52

(52) U.S. Cl. CPC A61F 2/52

(57) ABSTRACT

A silicone prosthesis that has elements for attachment to muscle or breast tissue, which does not move or modify the initial appearance, said silicone prosthesis being formed of a front surface (6) and a back surface (3), which form the material that covers the prosthesis (1), having elements for attachment in the form of protrusions (2), which are joined to the material that covers the prosthesis (1), wherein each of the protrusions (2) has a hole (4) through which a suture can be threaded and the protrusions (2) are distributed around the circumference (5) or along orthogonal axes on the front surface (6) of the base of the prosthesis (Ia). Another prosthesis model, in the form of a partial prosthesis (Ic), has a spherical half-cap shape and is provided with elements for attachment in the form of protrusions (2c). The holes (4) in the protrusions (2) can be provided with reinforcements (7).

This invention belongs to the technological sector of aesthetic and restorative plastic surgeries and refers, more specifically, to a silicone prosthesis that possesses elements for attachment to the muscle or breast tissue, to ensure that, over time, the prosthesis does not move and modify the initial appearance due to drooping.

### STATE OF THE ART

According to the International Society of Aesthetic Plastic Surgery (ISAPS), in 2013, a total of 23 million plastic surgeries were performed. Brazil occupies a prominent position in the survey as the country that performed the most surgical procedures, ahead of the U.S. with 1,491,721 of the total number. The U.S., however, still leads when the total volume of plastic surgeries—surgical and non-surgical—are considered. The second most commonly performed surgery in Brazil was the placement of breast prostheses.

Moreover, according to the International Society of Aesthetic Plastic Surgery (ISAPS), Brazil performed 1.22 million procedures in 2015, which means that despite a decline of almost 230,000 procedures per year compared to 2013, the country remains in second place in the ranking, surpassed only by the United States, which registered 1.41 million surgeries in 2015. The American Society for Aesthetic Plastic Surgery (ASAPS) recorded 305,856 augmentation mammoplasty procedures in the United States in 2015, where women aged between 19 and 34 years of age were the most frequent subjects of these procedures.

Breast reconstruction surgery is another procedure that can be performed for various reasons, but the principal and most common of them is the removal of one or both breasts to remove a tumor. The procedures, in these cases, serve to restore the appearance of the damaged area using reduction, augmentation or lifting techniques, but despite the variety of techniques available, most breast reconstruction surgeries use silicone prostheses.

In 2012, the American Society of Plastic Surgeons (ASPS) launched a national breast reconstruction awareness campaign to educate, engage and empower women to make informed decisions about their health after breast cancer diagnosis and mastectomy. In 2015, in the USA, the number of reconstruction procedures increased by 4%, totaling more than 106,300 cases, and since 2000, the numbers have increased by 35%.

It is known that breast cancer is the form of cancer that most affects women worldwide, both in developing and developed countries. About 1.67 million new cases of this neoplasm were projected for the year 2012 worldwide, representing 25% of all types of cancer diagnosed in women. Its rates of incidence vary in different regions of the world. The National Cancer Institute (INCA) estimates that for each year from 2018 to 2019, 59,700 new cases of breast cancer will be diagnosed in Brazil, with an estimated risk of 56.33 cases per 100,000 women.

In the light of this, it can be observed that the technology used in prostheses has great importance not only for the aesthetic procedures sector, but also in contributing to recovery in cancer cases.

That said, we can cite some alternatives that relate to the technologies used to improve silicone implant procedures and represent the current state of the art. Patent document No. EP 3085337-“PROSTHESIS FOR SUPPORTING A BREAST STRUCTURE” presents a prosthesis supporting part intended to act as a net and which should have sufficient mechanical properties to provide the breast implant with support against gravity and thus limit the risk of drooping in the breast implant. The attachment part provided by this invention is intended to be attached to the pectoral muscle. The attachment part is intended to face the upper portion of the breast structure and should demonstrate mechanical properties adapted to ensure the anchorage of the prosthesis to the pectoral muscle. The document in question resolves some of the problems described above, but does not present a complete solution, which, in addition to preventing the slippage of the prosthesis, also prevents lateral and rotary displacement. Moreover, it did not present constructive options where attaching the prosthesis by sewing is possible.

In patent document No. BR 1020150186266—“SILICONE BREAST PROSTHESIS WITH A MIXED POLYURETHANE COATING AND TEXTURIZED BASE SURFACE” provides a silicone breast prosthesis with a mixed polyurethane coating and textured base surface, which offers the possibility of safe implantation in any space, whether subglandular, subfascial or submuscular. The textured base of the mixed prosthesis prevents sticking to the costal arch, while most of the mixed prosthesis is composed of polyurethane that provides greater adhesion preventing early drooping of the breasts and displacement of the prosthesis. Despite providing a textured coating with polyurethane parts to avoid the movement of the prosthesis, this method uses friction between the polyurethane and the biological tissue of the patient, which still does not solve the problems described above, since the friction is restricted and can easily present flaws and slippage. Moreover, it did not present constructive options where attaching the prosthesis by sewing is possible.

U.S. Pat. No. 9,750,600, entitled “BREAST IMPLANT”, refers to a breast prosthesis that possesses two strips attached to the front surface and two strips to the back surface. Each of the strips has a proximal end attached to the surface of the prosthesis by a sealant, seal or adhesive. The distal end of the strips possesses a hole for threading the suture filament into the human body. This prosthesis is limited to resolving the risk of rotation of the prosthesis, and only partially. This is due to the fact that the strips are elongated, and they may become deformed or their distal ends may be displaced. This document does not provide means for preventing the displacement of the prosthesis along the vertical and horizontal axes. In addition, the implantation of the prosthesis is difficult, as it requires skill and effort on the part of the surgeon to perform the suture through the back surface.

In this context, from the analysis of the documents of the state of the art, it is noted that the silicone prostheses currently available and known to the market, do not provide a solution to the proposed problem regarding its attachment to the patient's body. That is, none of these prostheses allows for attachment to the breast or muscle tissue, thus preventing the vectorial and angular displacement of the prosthesis after it has been surgically inserted into the patient's body.

### Novelties and Objectives of the Invention

In order to overcome the problems of the state of the art highlighted above, the present patent aims to propose a solution to the principal problem of the continuance of visual quality over time following surgical and aesthetic procedures involving the implantation of silicone prostheses. Thus, the prosthesis of the invention prevents displacement and modification of the initial aesthetic effect due to the drooping of the skin a certain time after its implantation.

In this regard, the present invention provides elements for attachment in the form of protrusions that are attachable to the front surface of the prosthesis. These protrusions can be positioned on two orthogonal axes and/or on the circumference of the base, and are equipped with holes for the threading of suture filaments to attach the prosthesis to the muscle or breast tissue, depending on the surgical method chosen by the physician at the time of the surgery.

Optionally, the present invention also provides a partial or segmental prosthesis that is indicated for cases of removal of only one sector or segment of the breast (sectorectomies) due to breast cancer.

The invention completely prevents rotational movement, as well as displacement in vertical or horizontal directions during the use of the prosthesis. The invention also uses elements for attachment implanted around the prosthesis that facilitate the execution of the suture, dispensing with the need for suturing behind the prosthesis as in the state of the art. In addition, the invention uses elements for attachment with continuous connection to the surface of the prosthesis providing a third advantage which is to prevent possible damage to the connection points.

The prosthesis of the invention is particularly used in three situations:

A) in augmentation mammoplasties and after mastectomy breast reconstructions to prevent the displacement of prostheses both on the vertical and horizontal axis, thus preventing asymmetries, lateral displacement of the prostheses towards the armpits, or medially preventing symmastia (an unwanted effect where prostheses move excessively towards the center of the thorax, giving the impression of the breasts joining on the midline);

B) in mastopexies with a prosthesis, the attachment of the implant to the suprajacent breast tissue and chest by the attachment points prevents both the slipping of the prosthesis and the drooping of the breast tissue since, in many cases, even if the prosthesis remains in its proper position on the thorax, the breast tissue and the breast skin being flaccid, droop prematurely, and over time, due to the action of gravity, the aesthetic result initially achieved is prematurely lost. Thus, the prosthesis is attached to the thoracic wall by the radial attachment points and anchored to the breast tissue by the attachment points located on the front surface thereof. This adhesion of the prosthesis to the breast tissue, as if the implant were part of the gland, confers greater compression on the breast so that there is less tension on the cutaneous membrane and consequently greater durability of the shape of the breast and the texture of its skin;

C) in sectorectomies where, because of breast cancer, a segment of the compromised gland is removed (not the entire gland as in mastectomies) resulting in loss of volume and consequent asymmetry.

The partial or segmental prosthesis can be tailored to each case, on being implanted in the place where the tissue was removed, with the elements for attachment being anchored to the muscle or gland, thus restoring the shape and volume of the breast and avoiding the need to implant another prosthesis in the contralateral breast for sintering.

The female breast is associated with beauty, sexuality and femininity. In addition to its physiological role, it possesses great emotional importance. In this regard, the main reason that leads women to undergo an aesthetic procedure is to improve the shape or volume of the breast, when it is small or disproportionate to the body, and does not correspond to the desired self-image. This surgical procedure is also used to correct breast asymmetry. One of the most common cases is when a surgeon seeks to reconstruct a breast that has been resected for reasons related to cancer or to restore symmetry to the other breast, in the same situation.

Augmentation mammoplasty is a surgical procedure that typically requires local anesthesia with sedation or general anesthesia, and lasts approximately 2 hours. The surgeon begins by making an incision in the breast, through which the prosthesis will be inserted. Depending on the patient, the purpose or size of the implant, the incision may be made in the submammarian, periareolar or axillary area. The implant may be placed in the pectoral muscle (submuscular) or glandular tissue, in this case being placed under or above the muscle. The type of incision, as well as the location of insertion of the implant should be determined by the surgeon together with the patient, taking into account the clinical case and their expectations.

Similarly, mastopexy surgery or breast lifting, seeks to reshape and lift the breasts. The technique is used to reverse the natural drooping present in the region, known as breast ptosis. Drooping can be measured according to 4 classifications: Grade 1, mild, where the areola is at the level of the inframammary fold and above the circumference of the gland; Grade 2, moderate, the breast nipple is below the level of the inframammary fold, but above the circumference of the gland; Grade 3, accentuated, the areola is below the mammary fold and the circumference of the gland.

Mastopexy surgery may or may not be associated with a silicone prosthesis. The advantage of opting for a mastopexy with a prosthesis is that the implant will give more firmness to the breast. However, if a large prosthesis is inserted and the patient's skin does not have adequate density, the breasts may give and the drooping may return. But this association of mastopexy surgery with silicone produces very good results, because in addition to enlarging the breasts, it also improves the appearance of the bust in terms of shape and texture.

Immediate breast reconstruction is oncologically safe and has been indicated more frequently, provided there is an adequate histological study. In addition to the obvious psychological benefit, the preservation of body image is undoubtedly a strong reason for it to be encouraged. Reconstruction with prostheses is performed only with the insertion of a retromuscular silicone prosthesis to replace the removed breast tissue.

To this end, in order to improve the cited procedures, as well as other related techniques, the present invention presents an innovative silicone prosthesis, adaptable to any technique, which has elements for attachment in subglandular, submuscular and subfascial applications.

Also known as subglandular silicone prosthesis, it is the technique responsible for placing the implant below the mammary glands and over the muscle. This is one of the most common positions, especially for those who want to keep their breasts prominent. However, it is necessary to be aware of some details that may be harmful, such as if the silicone prosthesis is too large. In this case, over time, its weight can cause some sagging of the skin. Another important point for silicone prosthesis surgery over the muscle is whether the woman is too thin or has very thin skin. In this situation the rippling effect can occur, which is the slight visual perception of the texture of the prosthesis and, on observation, it looks as if the skin of the breasts is wrinkled.

And there is also the submuscular or retromuscular silicone prosthesis, where the plastic surgeon raises the pectoral muscle to insert the implant. This position is indicated for women who have thin skin, as they will not have the intense sensation of feeling the prosthesis. In addition, submuscular silicone is ideal for those seeking a more discreet appearance, without the cleavage being too prominent. Over time, there may be drooping of the breasts, but with this method the prosthesis does not subside, as it is anchored to the muscle. In some cases, this can cause the “double bubble” effect, where the silicone stays in place, but the skin gives way. A silicone prosthesis behind the muscle is also advisable for those who have a history of breast cancer. This is because the implant is further away from the mammary glands, where the nodules form. So, if necessary, it is possible to treat the disease without removing the silicone prosthesis.

Similarly, the subfascial or dual plane prosthesis, as it is also known, is the latest technique, where the plastic surgeon combines elements of the two aforementioned techniques. Women have two mammary muscles protected by a thin layer known as the fascia. Above this layer are the mammary glands, responsible for milk production and breastfeeding. On the subfascial plane, the implant is positioned on top of the mammary muscles, but under the fascia layer and glands. The fascial membrane serves as a support without affecting the shape, leaving the appearance of the natural silicone prosthesis clearer. In addition, this technique is less traumatic than silicone underneath the muscle.

### DESCRIPTION OF THE ATTACHED DRAWINGS

In order for this utility model to be fully understood and put into practice by any skilled person from this technology sector, it will be clearly, concisely and sufficiently described below, based on the attached drawings, which illustrate and support it:

FIG. 01—lower perspective view of the model of the prosthesis of the invention;

FIG. 02—lower perspective view of the model of the prosthesis;

FIG. 03—upper perspective view of a detail of an attachment element of the prosthesis;

FIG. 04—upper perspective view of another model of the prosthesis;

FIG. 05—upper perspective view of yet another model of the prosthesis;

FIG. 06—perspective view of a partial prosthesis of the invention;

FIG. 07—upper view of the partial prosthesis.

### DETAILED DESCRIPTION OF THE INVENTION

The prosthesis (1) of the invention is equipped with a front surface (6) and a back surface (3), being equipped with elements for attachment in the form of protrusions (2) that are attached to the cover material of the prosthesis (1), where each of the protrusions (2) is equipped with a hole (4) for the threading of the suture filament.

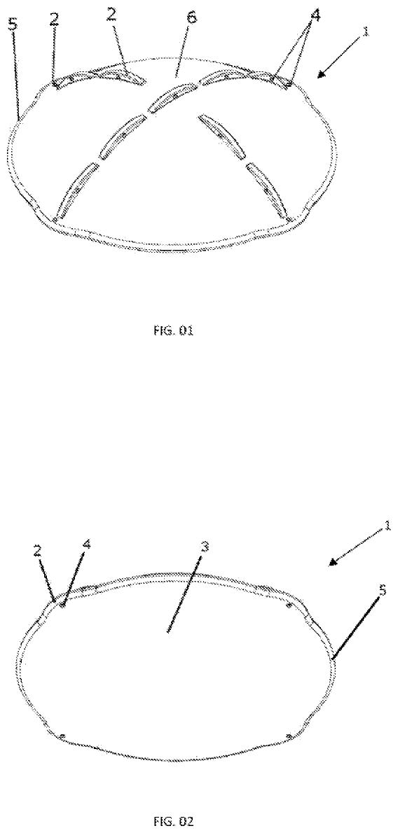

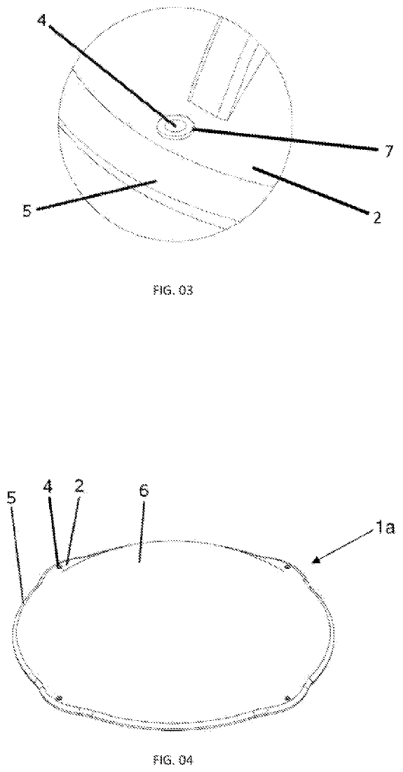

FIGS. 1 and 2 detail a model of the prosthesis (1) where the protrusions (2) are aligned on two orthogonal axes on the front surface (6) and also on the circumference (5) of the base of the spherical cap, and the protrusions (2) are equipped with holes (4) for the threading of suture filaments by the physician during surgery.

FIG. 3 details a protrusion (2) with a hole (4) that optionally may contain a supporting element (7), preferably Nylon, stainless steel or any other rigid material compatible with the human body. In one alternative modality, said support (7) may comprise a cylindrical body, with a height compatible with that of the hole (4) and two attachment rings that extend over the hole (4) of said prosthesis (1) to retain the support of the prosthesis (1), located in the lower part and in the upper part of said support (7). Through this hole (4) equipped with the support (7) of the protrusions (2) nylon threads are passed that serve to anchor the prosthesis (1) to the muscle tissue or breast tissue. The anchoring to the patient's biological tissue can be performed by sewing, a method already known to a person skilled in the art.

It is also worth highlighting that the elements for attachment in the form of protrusions (2) for the silicone prostheses, as proposed in the present invention, should form part of the body of the prosthesis, being attached to the material that covers the prosthesis to provide adequate resistance related to the rupturing of these protrusions (2) from the surfaces of the prosthesis (1).

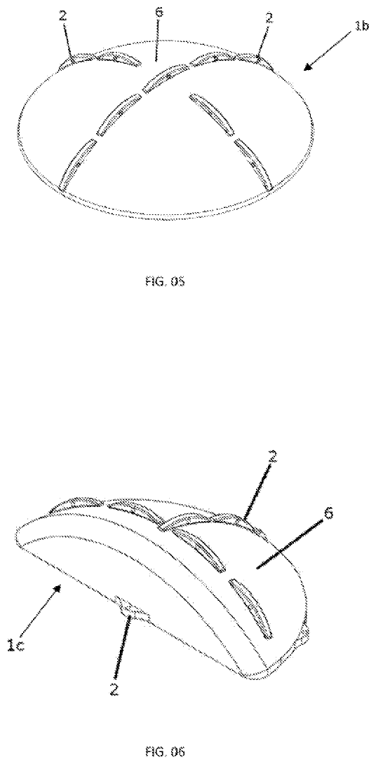

Example 1: in a first configuration, the present invention provides a silicone prosthesis (1a) where elements for attachment are arranged in the form of protrusions (2) only positioned on the circumference (5) of the base of the prosthesis, leaving its front surface (6) smooth. Said elements for attachment (2) may be arranged only on the cardinal points of the prosthesis, as exemplified in FIG. 04, so that the prosthesis can be attached with its base in the patient's biological tissue. By way of example, in prostheses that are introduced in patients using the subglandular technique, the elements for attachment of the profile (5), can be duly attached in the muscle tissue and thus prevent the rotation of the prosthesis and provide greater stability and safety of attachment thereof.

Example 2: in a second form of execution, the present invention provides a prosthesis (1b) where elements for attachment are distributed in the form of protrusions (2) only on its front surface (6). The protrusions (2) may be arranged in different positions on their front surface (6), as exemplified in FIG. 05 so that the prosthesis (1b) is attached and does not move or rotate and, specifically, keep the breast tissue anchored thereto preventing premature ptosis and loss of shape By way of example, with prostheses introduced into patients using the submuscular technique, the prosthesis (1b) with elements for attachment in the form of protrusions (2) on the front surface (6), can be anchored to muscle tissue via nylon threads, or any other compatible material, passing through the protrusions (2).

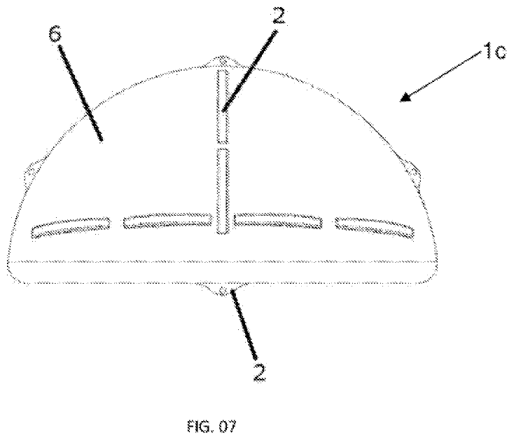

Example 3: in a third form of execution, the present invention provides a partial prosthesis (1c) in the form of a spherical half-cap, equipped with elements for attachment in the form of protrusions (2c), exemplified in FIGS. 6 and 7. This prosthesis (1c) can be implanted through a surgery where the entire breast is not removed, a common situation in people with breast cancer, where there is a need to remove only the cancerous part. This part can be replaced by a partial or segmental prosthesis (1c), which, through the protrusions (2), is attached by a suture, a method already known to persons skilled in the art. The advantage of a partial or segmental prosthesis (1c) is that one can reconstruct the breast affected by the cancer by restoring the symmetry of the breasts without the need to operate on the contralateral breast. In reconstructions with traditional prostheses, in most cases, bilateral implants of different volumes are required.

In one modality the silicone prosthesis equipped with elements for attachment is implanted in the biological tissue.

In one modality the silicone prosthesis is used to replace biological tissue.

In one modality, the biological tissue belongs to an individual with cancer or a healthy individual.

It is important to point out that the description provided is not intended to limit the forms of execution of the inventive concept proposed here, but rather to illustrate and make comprehensible the conceptual innovations revealed in this solution. Thus, the descriptions must be interpreted in an illustrative and non-limiting manner, and there may be other equivalent or analogous forms of implementation of the inventive concept hereby revealed which are not excluded by the spectrum of protection described in the proposed solution.

This specification provides a new silicone prosthesis arrangement with handles for attachment in the muscle or breast tissue, or in other muscles that have similar anatomy, for example, in the buttocks, which possesses novelty, inventive step, descriptive sufficiency, industrial application and which, consequently, fulfills all the essential requirements for the granting of the privilege.

1. A Silicone prosthesis equipped with elements for attachment comprising a front surface and a back surface which form the material that covers the prosthesis and elements for attachment, characterized by having at least three elements for attachment in the form of thick corpuscles incorporated into the circumference of the base of the material that covers the prosthesis, leaving its front surface smooth and having in each of the thick corpuscles at least one hole for the threading of the suture filament.

2. The silicone prosthesis of claim 1 wherein the elements for attachment further comprises at least four elements for attachment incorporated into the front surface of the material that covers the prosthesis and by the elements for attachment having the form of thick corpuscles with at least one hole for the threading of the suture filament.

3. The silicone prosthesis of claim 1 characterized by the holes of the thick corpuscles being equipped with supports.

4. The silicone prosthesis of claim 1, characterized by said support comprising a cylindrical body, with a height compatible with that of the hole and two attachment rings that extend over the hole of the prosthesis located in the lower and upper parts of said support.

5. The silicone prosthesis of claim 2 characterized by the thick corpuscles distributed on its front surface being aligned on two orthogonal axes.

6. The silicone prosthesis of claim 1 wherein the prosthesis is implanted in biological tissue.

7. A Silicone prosthesis equipped with elements for attachment comprising a front surface and a back surface which form the material that covers the prosthesis and elements for attachment, characterized by having at least four elements for attachment in the form of thick corpuscles incorporated into the front surface of the material that covers the prosthesis and each of the thick corpuscles having at least one hole for the threading of the suture filament.

8. The silicone prosthesis of claim 7 wherein the elements for attachment further comprises at least three elements for attachment incorporated into the circumference of the base of the material that covers the prosthesis.

9. The silicone prosthesis of claim 7 characterized by the holes of the thick corpuscles being equipped with supports.

10. The silicone prosthesis of claim 7, characterized by said support comprising a cylindrical body, with a height compatible with that of the hole and two attachment rings that extend over the hole of the prosthesis located in the lower and upper parts of said support.

11. The silicone prosthesis of claim 7 characterized by the thick corpuscles distributed on its front surface being aligned on two orthogonal axes.

12. The silicone prosthesis of claim 7 wherein the prosthesis is implanted in biological tissue.

13. A Silicone prosthesis equipped with elements for attachment comprising a front surface and a back surface forming the covering material of the partial prosthesis which presents the form of a spherical half-cap and elements for attachment, characterized by having at least three elements for attachment incorporated into the circumference of the base of the material that covers the prosthesis and at least three elements for attachment incorporated into the front surface of the material that covers the prosthesis and by the elements for attachment having the form of thick corpuscles with at least one hole for the threading of the suture filament.

14. The silicone prosthesis of claim 13 characterized by the holes of the thick corpuscles being equipped with supports.

15. The silicone prosthesis of claim 13 according to claim 14, characterized by said support comprising a cylindrical body, with a height compatible with that of the hole and two attachment rings that extend over the hole of the prosthesis located in the lower and upper parts of said support.

16. The silicone prosthesis of claim 13 characterized by the thick corpuscles distributed on its front surface being aligned on two orthogonal axes.

17. The silicone prosthesis of claim 13 wherein the prosthesis is implanted in biological tissue.

18. A method of use of the silicone prosthesis of claim 1 comprising the step of implanting the prosthesis into biological tissue.

19. A method of use of the silicone prosthesis of claim 7 comprising the step of implanting the prosthesis into biological tissue.

20. A method of use of the silicone prosthesis of claim 13 comprising the step of implanting the prosthesis into biological tissue.
